(19) United States
(12) Patent Application Publication
(10) Pub. No.: US 2006/0147500 A1
(43) Pub. Date: Jul. 6, 2006

(54) Use of isomalt (mixture of 1,6 gps and 1,1 gpm) as a prebiotic for the production of a medicament used for the treatment of intestinal diseases, among other things

(76) Inventors: Michael Klingeberg, Grunstadt (DE); Gunhild Kozianowski, Grunstadt (DE)

Correspondence Address: OSTROLENK FABER GERB & SOFFEN, 1180 AVENUE OF THE AMERICAS, NEW YORK, NY, 100368403, US

(21) Appl. No.: 10/561,122

(22) PCT Filed: Jun. 4, 2004
(86) PCT No.: PCT/EP04/06030

§ 371 Date: Feb. 2, 2006

(30) Foreign Application Priority Data
Jun. 16, 2003 (DE) 103 28 180.0

(51) Int. Cl. A61K 31/7012; A61K 31/715; A23K 1/165

(52) U.S. Cl. 424/442; 514/25; 514/54

(57) ABSTRACT

The present invention relates to a novel use of a mixture of 6-O-α-D-glucopyranosyl-D-sorbitol and 1-O-α-D-glucopyranosyl-D-mannitol.

The present invention relates to the use of a mixture of 6-O-α-D-glucopyranosyl-D-sorbitol (1,6-GPS) and 1-O-α-D-glucopyranosyl-D-mannitol (1,1-GPM) as prebiotic and/or butyrate-supplying, partially digestible and intestinal health-promoting carbohydrate in human food and other consumable products, animal feed products, and/or medicaments.

Human food and other consumable products and animal feed products are primarily used for the nutrition and the wellbeing of the human or animal consumer. Besides these two aspects, human food and other consumable products are increasingly also expected to have a health-promoting function. Human food and other consumable products should on the one hand maintain and promote health, and on the other hand fend off harmful influences and, where appropriate, act prophylactically against diseases. Such health-promoting human food and other consumable products are intended to display their effect predominantly in the digestive tract. Consumed foodstuffs are broken down and partly absorbed in the anterior digestive tract. Indigestible carbohydrates reach the large intestine and are available to the microbial intestinal flora there. This intestinal flora includes bacteria such as Bacteroides, Eubacterium, Bifidobacterium, Lactobacillus, Atopobium and Fusobacterium. Besides these, Escherichia coli and microorganisms which are facultative pathogens, such as clostridia, staphylococci and other enterobacteriacea, occur. Lactobacteria, especially bifidobacteria, are known to have health-promoting properties. They produce to a large degree short-chain organic acids and inhibitors which limit the growth and the activity of harmful bacteria which form unwanted enzymes such as β-glucosidases, β-glucuronidases or azoreductases. The importance of some unwanted bacterial enzymes such as β-glucosidases derives from the formation, activation and liberation of toxic, carcinogenic and cocarcinogenic compounds from endogenous and exogenous substances. For example, bacterial β-glucosidase liberates toxic aglycones from glycosides. Inhibition of harmful bacteria and thus inhibition of the activity of bacterial enzymes such as β-glucosidase limits the production of endotoxins and carcinogenic compounds, and improves the excretion of xenobiotics. A further beneficial property of the health-promoting intestinal flora comprises immunomodulatory effects and immune function stimulation. Lactobacteria, especially bifidobacteria, additionally have, through inhibition of harmful and pathogenic bacteria, a protective and preventive effect in relation to intestinal infections, especially bacterial diarrheas.

Short-chain fatty acids such as butyric acid (butyrate) are formed in particular from undigested carbohydrates by fermentation by saccharolytic bacteria in the large intestine. Butyric acid is the dominant energy source for the epithelial cells in the colon, influences cellular proliferation and differentiation and plays a central part as growth factor for a healthy intestinal epithelium and in maintenance of the mucosal barrier in the colon. Short-chain fatty acids such as butyric acid or its salts, butyrate, contribute to the detoxication of possible mutagenic metabolites in the large intestine and counteract oxidative stress, for example through induction of gene expression of protective proteins such as intestinal glutathione S-transferase or inhibition of ornithine decarboxylase. In addition, short-chain fatty acids such as butyric acid have a controlling effect on the induction of specific genes and the modification of proteins in regulation of the cell cycle, antibacterial peptides and signal cascades. High butyric acid concentrations in the large intestine, especially in the posterior regions of the large intestine, support a healthy intestinal milieu and a healthy intestinal epithelium, improve symptoms of ulcerative inflammations in the colon and are protective in colon carcinogenesis, meaning that they are regarded as reducing the risk of cancer of the large intestine.

It is desirable to promote the intestinal flora which have a beneficial effect on human or animal health and, in addition, to achieve a production of large amounts of butyric acid, especially in the posterior sections of the large intestine too. This can be achieved by supplying suitable substrates for improving the living conditions for the health-promoting intestinal flora, and substrates for the microbial formation of butyric acid, also in posterior regions of the large intestine.

Substances or mixtures of substances which, as constituents of human food or other consumable products, selectively promote the growth and/or the activity of specific health-promoting intestinal bacteria, especially bifidobacteria and lactobacilli, are referred to as prebiotics. Prebiotics promote the growth and/or the activity of health-promoting intestinal bacteria and are usually carbohydrates which cannot be digested by enzymes of the gastrointestinal tract.

Cummings et al. (Am. J. Clin. Nutr. 73 (2001), 415-420) disclose that prebiotics may be long-chain carbohydrates, for example inulin or fructooligosaccharides. Kummel & Brokx (Cereal Foods World 46 (2001), 424-429) describe the prebiotic lactitol. However, long-chain carbohydrates which cannot stimulate the growth of bifidobacteria are also known. These include higher molecular weight vegetable hemicelluloses such as xylan from larches, wheat and oats or polysaccharides of marine origin such as laminarin and alginate. Said polysaccharides are metabolized mainly by the genus Bacteroides.

In turn, not all saccharides known to be prebiotics serve as suppliers of butyrate and, if they do, it is only in the anterior regions of the large intestine. Known prebiotics such as fructooligosaccharides which reach the large intestine are fermented there quite quickly and completely. The short-chain fatty acids formed in this case are absorbed rapidly and almost completely by the intestinal epithelial cells at the site of their production. However, to supply butyrate in the posterior sections of the intestine it is necessary for the saccharides to be fermented more slowly, so that substrate also reaches and is available for microbial butyrate formation in regions of the intestine located posteriorly. A very rapid fermentation of known prebiotics may also mean inter alia an increased risk of laxative effects and other gastrointestinal upsets.

A further disadvantage of known prebiotics such as inulin and oligofructose is that predominantly other short-chain fatty acids, especially acetic acid, are formed when they are broken down by the intestinal microflora, and they therefore supply butyric acids to only a very small extent. Known prebiotics such as fructooligosaccharides also have the disadvantage that their technological processibiity during food manufacture is unsatisfactory in some cases. Poor solubility in water, for example of long-chain carbohydrates such as resistant starch, their low stability to acid and their reactivity as, in some cases, reducing oligosaccharides contribute to their limited usability. This applies especially on use in products having a low pH. A further disadvantage of known prebiotics is that they are fermented comparatively quickly and/or lead to only little butyric acid formation and thus have only low butyrogenicity.

The present invention is therefore based on the technical problem of providing substances or mixtures of substances which are able to undertake a prebiotic function in human food and other consumable products, animal feed products, and medicaments and, at the same time, assist butyric acid formation and also overcome the above-mentioned disadvantages, in particular which act as bifidogenic prebiotics and serve as butyrate-supplying (butyrogenic) substrate with technological processibility which is as good as possible, advantageous nutritional characteristics and good tolerability.

The present invention solves the technical problem on which it is based through the provision of a use of mixtures of 1,6-GPS and 1,1-GPM in human food and other consumable products, animal feed products, and medicaments as prebiotic, in particular prebiotic having a bifidogenic effect and/or as fermentable substrate, in particular as butyrate-supplying substrate which is at the same time slowly fermentable, with good technological processibility.

The present invention is based inter alia on the fact that the mixture of 1,6-GPS and 1,1-GPM, employed in human food and other consumable products, animal feed products, and medicaments, has a prebiotic, in particular bifidogenic activity and/or serves as substrate for butyric acid formation after consumption in the gastrointestinal tract of the human or animal consumer.

Investigations on the mixture used according to the invention surprisingly revealed that consumption of a mixture of 1,6-GPS and 1,1-GPM leads to a multiplication of good and health-promoting bacteria, especially bifidobacteria, in the intestinal tract of the consumer, as can be demonstrated for example by an increase in the bifidobacteria in the stool flora. The mixture used according to the invention is additionally notable for consumption also leading to an increase in the proportion of bifidobacteria in the total flora in the consumer.

It was also evident from the investigations that consumption of the mixture used according to the invention also contributes to a beneficial influence on the activity of the microflora, especially through reducing the activity of the bacterial enzyme β-glucosidase, which leads to the formation of toxic compounds in the intestine.

A further evident advantage is that the mixture employed according to the invention can be consumed with the daily diet in relatively large quantities, for example 30 g/d and more, without unpleasant gastrointestinal occurrences.

The bifidogenic and prebiotic effect of the mixture used according to the invention was surprising insofar as virtually no reduction in pH by bifidobacteria strains was observed with 1,6-GPS or 1,1-GPM in earlier in vitro investigations, and this did not initially indicate that isomalt has a bifidogenic or prebiotic property (Kashimura et al., 1991).

By contrast, it has now been possible in the context of the present invention to show that bifidobacteria grow with isomalt and are able to degrade it and moreover form short-chain fatty acids. It has further been possible to show that the mixture used according to the invention is also suitable as, preferably sole, source of carbon and energy for bifidobacteria.

In this connection, “bifidobacteria” or “bifidus flora” means a genus of Gram-positive, non-motile, non-sporing and anaerobic rod bacteria which mainly colonize the large intestine, especially of the species B. adolescentis, B. bifidum, B. breve, B. catenulatum, B. longum and B. infantis. They cleave carbohydrates to form short-chain organic acids, especially acetic acid (acetate) and lactic acid (lactate). The pH of the surroundings is reduced thereby, and inhibition of pathogenic bacteria becomes possible. Bifidobacteria are bacteria regarded as particularly desirable for human health. The beneficial effects of bifidobacteria include suppression of pathogenic microbes, reduction in the concentration of ammonia and lipids in the blood, regeneration of intestinal flora damaged by antibiotics, stimulation of the immune system and an immunomodulatory effect, for example also for assisting with defense against malignant cells, and the production of vitamins such as B vitamins and folic acid. Bifidobacteria are regarded as important carriers of the resistance to colonization by pathogenic bacteria and as antagonists of the putrefactive flora. They contribute, through the fermentative production of short-chain fatty acids in the large intestine and inhibitors, to inhibition of the growth of harmful bacteria and their activity, for example by inhibiting harmful bacterial enzymes such as β-glucosidase. By inhibiting pathogenic bacteria, bifidobacteria also have a protective and preventive effect against infections, especially bacterial intestinal infections. Bifidobacteria contribute, through the production of short-chain fatty acids in the large intestine, to the supply of nutrients and maintaining the health of the large intestinal mucosa.

Unlike lactobacilli, for example, the use of bifidobacteria is, because of their sensitivity to atmospheric oxygen, impossible or possible to only a limited extent in food products, i.e. probiotic food products. It is possible by combining probiotic cultures and the mixture used according to the invention as a prebiotically acting substance to achieve improved survival of the living bacteria in synbiotic products, and the stimulation both of consumed and, in particular, of endogenously present beneficial bacteria such as bifidobacteria in the entire intestinal tract.

It has been possible to show further that the mixture used according to the invention is additionally metabolized by human intestinal flora by slower fermentation and, during this, leads to a higher butyrate production than for example the known prebiotic fructans.

The slower fermentation of the mixture employed according to the invention compared with known prebiotic substrates and the simultaneously greater formation of butyric acid results in the mixture used according to the invention and consumed with the diet also reaching to a far larger extent the posterior regions of the large intestine and being able to serve there as active substance, for example for the treatment or prevention of large-intestinal disorders.

The mixture employed according to the invention is further notable for an extremely good technological processibility in human food and other consumable products and animal feed products, also because of its solubility in water and stability to acid. The stability to acid makes the use according to the invention suitable in particular for products having a low pH.

The use according to the invention of said mixture is also advantageously notable for being usable in humans and animals for supporting and stabilizing a healthy intestinal flora, for promoting a healthy metabolism by the intestinal flora, for maintaining a healthy intestinal epithelium, for supporting intestinal health, for reducing toxic and harmful intestinal contents, for the prevention and treatment of chronic inflammatory bowel disorders and/or for preventing intestinal cancer and other disorders of the intestinal epithelium. The mixture can additionally be used for the prevention and control of infectious diseases, especially including bacterial intestinal infections and diarrheas, and for modulation and support of the immune system, as substance with properties of soluble dietary fibers and/or substance having prebiotic properties.

These beneficial effects of the inventive use of the mixture employed on the health of humans and animals are also attributable to the increase in the quantity and the proportion of lactobacteria, especially bifidobacteria, in and on the intestinal flora, the inhibition of harmful bacterial enzymes such as β-glucosidase and/or the slower fermentation and simultaneously high butyric acid formation by the intestinal flora.

The mixture employed in the use according to the invention advantageously reaches the large intestine, where it then serves as substrate for the microorganisms present there, such as lactobacteria, especially bifidobacteria, and is fermented to short-chain fatty acids. There is moreover a stimulation of the bifidobacteria and an increase both in the number of bifidobacteria and in the proportion of the bifidobacteria in the total flora, and thus a shift in the flora toward a bifidus flora. The short-chain fatty acids produced by the bifidobacteria, and inhibitors result in an inhibition of the harmful bacteria and their activity, as shown in particular also by the reduction in the activity of microbial β-glucosidase which liberates toxic and carcinogenic compounds. The mixture of the invention therefore has bifidogenic and prebiotic properties. The isomalt of the invention is additionally fermented comparatively slowly by the human intestinal flora and promotes the saccharolytic microflora. High butyric acid concentrations in the large intestine support a healthy intestinal milieu, improve symptoms of ulcerative inflammations of the colon and are protective in colon carcinogenesis. Butyric acid acts as growth factor for a healthy intestinal epithelium and as substrate for the colonic cells and thus inter alia counteracts the development and growth of colon carcinomas. Butyric acid contributes to the detoxication of possible mutagenic metabolites in the large intestine and counteracts oxidative stress, for example by inducing protective proteins such as intestinal glutathione S-transferase or inhibition of ornithine decarboxylase. A healthy intestinal milieu prevents adverse effects such as diarrhea, constipation, inflammations and passage of unwanted substances and bacteria from the intestinal lumen into the body.

The use according to the invention of the mixture employed has a beneficial effect on the health of humans and animals, especially through increasing the quantity and the proportion of the lactobacteria, especially bifidobacteria, in and on the intestinal flora, and the slower fermentation and simultaneously high butyric acid formation by the saccharolytic intestinal flora. The use according to the invention of the employed mixture serves in humans to support and stabilize a healthy intestinal flora, to promote a healthy metabolism by the intestinal flora, to maintain a healthy intestinal epithelium, to maintain intestinal health, to reduce toxic and harmful intestinal contents, to reduce oxidative stress, to prevent and treat chronic inflammatory bowel disorders, prevent intestinal cancer, in particular large intestinal cancer also in posterior regions of the intestine and other disorders of the intestinal epithelium. The mixture additionally serves to prevent and control infectious diseases, especially including bacterial intestinal infections and for modulating and supporting the immune system.

In connection with the present invention, a “prebiotic” means an ingredient of human food and other consumable products, animal feed products or medicaments which selectively stimulates the growth and/or the activity of specific bacteria in the human or animal digestive tract, especially bifidobacteria and/or lactobacilli, so that health-promoting effects are to be expected. Prebiotics can usually be digested only with difficulty or not at all.

In connection with the present invention, a “probiotic” means a live microbial ingredient of a human food or other consumable product, animal feed product or medicament which promotes the health of the human or animal consumer by stabilizing or improving the microbial composition in the digestive tract. Examples of such probiotic microorganisms which can be employed in human food products, medicaments or animal feed products are: bifidobacterium such as the strains B. adolescentis, B. animalis, B. bifidum, B. longum, B. thermophilum; Enterococcus; Lactobacillus such as the strains Lb. acidophilus, Lb. brevis, Lb. casei, Lb. cellobiosus, Lb. crispatus, Lb. delbrueckii subsp. Bulgaricus, Lb. fermentum, Lb. GG, Lb. johnsonii, Lb. lactis, Lb. plantarum, Lb. reuteri, Lb. rhamnosus, Lb. salivarius; Bacillus cereus toyoi; Bacillus cereus, Leuconostoc; Pediococcus acidilactici; Propionibacterium; Streptococcus such as the strains S. cremoris, S. infantarius, S. intermedius, S. lactis, S. salivarius subsp. thermophilus (compare Fuller, J. Appl. Bacteriol. (1989)). Preferred probiotics are bacteria of the genera Lactobacillus and Bifidobacterium.

In connection with the present invention, “symbiotic” means a mixture of at least one prebiotic and at least one probiotic which, by improving the survival rate and increasing the number of health-promoting live microbial organisms in the gastrointestinal tract, promotes the health of the human or animal consumer, in particular by selective stimulation of the growth and/or the metabolic activity of the microbial organisms.

“Human food product” and “animal feed product” means substances or mixtures of substances which are used predominantly for human or animal nutrition and are in solid, liquid, dissolved or suspended form. An other consumable product means a substance or mixture of substances which are used predominantly for the pleasure derived by the human or animal body on consumption and are in solid, liquid, dissolved or suspended form. A medicament means substances or mixtures of substances which are used predominantly for the prophylaxis or therapy of diseases, impairments, injuries or manifestations of age of the human or animal body and are in solid, liquid, dissolved or suspended form.

In connection with the present invention, “disease” or “disorder” means an impairment of the vital processes and/or deficiency states in organs or in the whole body which is associated with a subjectively perceived and/or an objectively detectable physical and/or psychological change.

In connection with the present invention, “active substance” means a substance which can have a biological effect in living organisms or parts thereof. In this connection, this active substance may be used in particular to prevent, alleviate, cure or diagnose a disease. A “therapeutic active substance” means a substance which is used for the prevention or prophylaxis, alleviation or cure of a disease.

In connection with the present invention, “medicament” means a formulation of active substances which is intended for use on humans or animals.

The invention relates in a preferred embodiment to a use where the mixture of 1,6-GPS and 1,1-GPM in this use is isomalt. In connection with the present invention, isomalt means the mixture of 1,6-GPS and 1,1-GPM which is also referred to as Palatinit, for example a mixture which comprises from 43 to 57% by weight 1,6-GPS and from 57 to 43% by weight 1,1-GPM, based on the dry matter in the mixture.

In a further preferred embodiment of the present invention, the mixture employed according to the invention consists of 1,6-GPS and 1,1-GPM, consists substantially thereof or comprises these. The mixture is preferably a 1,6-GPS-enriched or a 1,1-GPM-enriched mixture or comprises this, as described in DE 195 32 396 C2 which, in relation to the preparation and composition of 1,6-GPS- and 1,1-GPM-enriched mixtures, is completely included in the disclosure of the present teaching.

In a further preferred embodiment of the present invention, the mixture of 1,6-GPS and 1,1-GPM employed according to the invention in a human food or other consumable product, animal feed product, or medicament is present as sole prebiotic and/or as sole butyrogenic substrate and/or as sole sweetener in the human food or other consumable product, animal feed product, or medicament. It is, of course, also provided for the mixture of 1,6-GPS and 1,1-GPM to comprise further substances or mixtures of substances, for example 1,1-GPS (1-O-α-D-glucopyranosyl-D-sorbitol). The mixture employed according to the invention may, besides 1,6-GPS and 1,1-GPM, also comprise mannitol, sorbitol, hydrogenated or non-hydrogenated oligosaccharides.

A further preferred embodiment provides for the mixture of 1,6-GPS and 1,1-GPM which is employed according to the invention as prebiotic and/or as butyrogenic substrate to be employed in the target products, meaning the human food or other consumable products, animal feed products, or medicaments, together with at least one further soluble and/or at least one insoluble, fermentable or non-fermentable dietary fiber and/or non-digestible carbohydrate.

Examples of soluble and/or insoluble fibers which are provided are: polydextrose; fructooligosaccharides having short and long saccharide chains, for example β(2→1)fructans, for example from the extraction from chicory root, and possible subsequent partial hydrolysis, or from transfructosylation of sucrose; galacto-oligosaccharides and transgalactosylated oligosaccharides, for example by transgalactosylation of lactose such as 6′-galactosyllactose (with Aspergillus oryzae β-galactosidase) or 4′-galactosyl-lactose (with Cryptococcus laurentii or Bacillus circulans β-galactosidase); partially hydrolyzed guar gum, such as “Sunfibre” or “Benefibre”; lactulose; lactitol; maltitol; sorbitol; mannitol; xylitol; erythritol; hydrogenated starch hydrolysates; xylo-oligosaccharides, for example having β(1→4) linked xylose units, for example from the enzymatic hydrolysis of xylan; Xylo-Gold from Meneba or xyloarabans; lactosucrose; malto-oligosaccharides such as “Fiber-sol-2” from Matsutani, and isomalto-oligosaccharides, such as from Showa Sangyoi, for example from the transgalactosylation of maltose; for example having α(1→4)-glucose linked via α(1→6)-glucose; gentio-oligosaccharides, for example oligosaccharides having β(1→6)-links; pyrodextrin, for example from the pyrolysis of corn or potato starch; glucosylsucrose, such as “Coupling sugar” from Hayashibara; soybean oligosaccharides, such as mixtures of raffinose (Gal-Glc-Frc) and stachyose (Gal-Gal-Glc-Frc) from the extraction of soybean whey; chito-oligosaccharides or chitosan-oligosaccharides; di- and oligosaccharides from honey, pectins and oligosaccharides obtained from pectins, also by partial hydrolysis; condensed oligosaccharides, for example from the condensation of saccharides, also saccharides modified by enzymatic modification and hydrogenation; di- and oligosaccharides obtained by caramelization of saccharides; galactomannan-oligosaccharides, carbohydrates with other monosaccharides; carbohydrates with other monosaccharides, di- and oligosaccharides for example also obtained by partial hydrolysis or oxidation or other modification of di- and oligosaccharides. It is preferred according to the invention to use at least one dietary fiber and/or non-digestible carbohydrate which is a fructo-oligosaccharide, polydextrose, inulin, a galacto-oligosaccharide, lactulose, lactitol, a xylo-oligosaccharide, lacto-sucrose, a malto-oligosaccharide, an isomalto-oligosaccharide, a gentio-oligosaccharide, glucosylsucrose, a soybean oligosaccharide, a chito-oligosaccharide, a chitosan-oligosaccharide, a pectin, a condensed oligosaccharide, a caramel product, a galactomannan-oligosaccharide, a fucose-containing oligosaccharide, a fucose derivative-containing oligosaccharide, modified starch, partially hydrolyzed guar gum, maltitol, sorbitol, mannitol, xylitol, erythritol, hydrogenated starch hydrolysate, pyrodextrin or a variant obtained by partial hydrolysis, hydrogenation, oxidation, enzymatic, chemical or other modification of saccharides. Resistant starches such as “Neo-Amylose” or “Actistar”, fiber materials from oats, wheat, vegetables, for example tomato or pea, fruits, for example apple, various berries, fruits of the carob tree, fiber materials from sugar beet, such as “Fibrex” from Danisco, from fruits of the locust tree, such as “Caromax” from Nutrinova, or cellulose or Vitacel from Rethenmaier.

In a further preferred embodiment of the present invention, the mixture of 1,6-GPS and 1,1-GPM employed according to the invention, where appropriate mixed with one of the aforementioned dietary fibers, in particular substances having a prebiotic and/or butyrogenic action, additionally comprises at least one probiotic, for example bacteria of the genus Lactobacillus and/or bifidobacterium, for example Bacillus cereus toyoi; Bacillus cereus; Bifidobacterium such as the strains: B. adolescentis, B. animalis, B. bifidum, B. longum, B. thermophilum; Enterococcus; Lactobacillus such as the strains Lb. acidophilus, Lb. brevis, Lb. casei, Lb. cellobiosus, Lb. crispatus, Lb. delbrueckii subsp. Bulgaricus, Lb. fermentum, Lb. GG, Lb. johnsonii, Lb. lactis, Lb. plantarum, Lb. reuteri, Lb. rhamnosus, Lb. salivarius; Leuconostoc; Pediococcus acidilactici; Propionibacterium; Streptococcus such as the strains S. cremoris, S. infantarius, S. intermedius, S. lactis, S. salivarius subsp. Thermophilus (compare Fuller, J. Appl. Bacteriol. (1989)), especially bacteria of the genus Lactobacillus and/or Bifidobacterium.

The mixture of 1,6-GPS and 1,1-GPM is therefore employed according to the invention in a particularly preferred embodiment as constituent of a synbiotic. It is possible through the combination provided by the invention of a probiotic and of the mixture employed according to the invention, in particular isomalt, as prebiotic to achieve advantageously a better survival of the probiotic bacteria during passage through the upper gastrointestinal tract and an improved success rate in the colonization of the probiotic bacteria in the intestinal tract, especially large intestine. In addition, the mixture having a prebiotic action which is employed according to the invention increases the growth and the activity both of exogenously supplied probiotic and of endogenously present bacteria, especially bifido bacteria.

In a further preferred embodiment of the present invention, the mixture of 1,6-GPS and 1,1-GPM employed according to the invention in a human food or other consumable product, animal feed product, or medicament is preferably used as prebiotic, in particular bifidogenic prebiotic and/or as butyrogenic, slowly fermentable substrate. In addition, a higher concentration of butyric acid (butyrate) is obtained through activation of saccharolytic bacteria in the large intestine through the butyrogenic and more slowly fermented mixture employed according to the invention, in particular isomalt.

It is of course possible for this mixture to comprise further additives and auxiliaries such as preservatives, colorings, flavorings, aromatizing substances, food-compatible acids, intensive sweeteners, emulsifiers, lubricants and release agents, medicinally active substances, vitamins, coenzymes, minerals or trace elements.

In a further preferred embodiment of the present invention, the mixture employed according to the invention is employed in human food products such as milk products, such as cheese, butter, yogurt, drinking yogurt, kefir, quark, sour milk, buttermilk, cream, condensed milk, dry milk, whey, milk sugar, milk protein, flavored milk, half-fat milk, flavored whey, or milk fat products or preparations; bakery products, in particular bread, rolls, croissants, including patisserie products or fine bakery products including preserved bakery products, cookie products or waffles; sandwich spreads, margarine products or baking fats; instant products and stock products; fruit products or fruit preparations such as jams, marmalades, jellies, preserving sugars, fruit conserves, fruit pulps, fruit puree, fruit juices, fruit juice concentrates, fruit nectar or fruit powders; vegetable products or preparations such as vegetable conserves, vegetable juices or vegetable puree; spice mixtures; muesli or muesli mixtures, and finished muesli-containing products; non-alcoholic beverages such as sports drinks and lemonades, beverage base materials and beverage powders; confectionary products such as chocolate, hard caramels, soft caramels, chewing gum, sugar-coated candies, fondant products, jelly products, licorices, marshmallow products, flaked products, compressed products, candied fruits, praline, nougat products, Eiskonfekt, marzipan, muesli bars, and ice cream or alcoholic and non-alcoholic sweet drinks, etc. and/or enteral nutrition forms.

A further preferred aspect of the present invention is the use of the mixture employed according to the invention as active substance, in particular as therapeutic active substance, in particular in medicaments, medicament-like preparations, human food and/or other consumable products and as addition in animal feed products for the treatment of disorders. These are in particular pharmaceutical compositions, a medicament comprising the isomalt according to the invention, and the use of the isomalt according to the invention for producing such medicaments. In one variant, the mixture employed according to the invention is used as active substance for the treatment of bowel disorders.

In further variants of the invention, the mixture employed according to the invention is used as active substance for the restoration and stabilization of a healthy intestinal flora, for the restoration and/or promotion of a healthy metabolism of the intestinal flora, for the restoration and/or promotion of a healthy intestinal epithelium, for the restoration and/or promotion of intestinal health, for the reduction of oxidative stress, for the reduction of toxic and harmful intestinal contents, for the prevention and/or treatment of chronic inflammatory bowel disorders, for the prophylaxis of intestinal cancer, especially large intestinal cancer, for the prophylaxis of infectious diseases, for the prophylaxis of bacterial intestinal infections and/or for the modulation and strengthening of the immune system.

The mixture employed according to the invention is additionally employed in particular also in animal feed products, both in the small-animal and in the large-animal sector.

The invention also relates to the use of the mixture employed according to the invention as active substance, where appropriate together with at least one of the aforementioned additives and auxiliaries, such as further prebiotics or non-digestible carbohydrates, in particular dietary fibers or substances having a fiber-like action, or probiotics, in a medicament or for producing a medicament for the control and/or prophylaxis of pathological states, impairments, injuries or manifestations of aging, especially also disorders and impairments of the gastrointestinal tract, of the human or animal body.

The mixture employed according to the invention is employed alone or, preferably, with other substances together in the human food or other consumable products, animal feed products, or medicaments in solid, for example crystalline but also amorphous, ground or liquid, in particular suspended or dissolved form. Suitable suspending agents or solvents are food-compatible solvents, especially water, alcohols and mixtures thereof.

Further advantageous configurations of the present invention are evident from the dependent claims.

The invention is explained in more detail by means of the following examples and relevant figures.

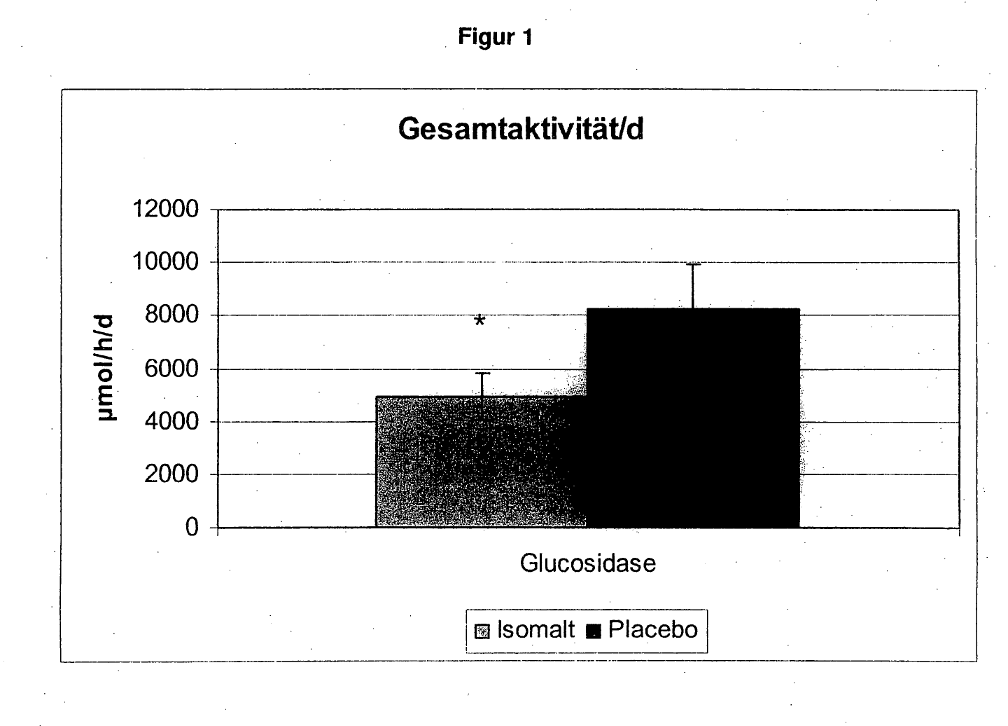

FIG. 1 shows the comparison of the total activity of microbial β-glucosidase in stool samples from subjects consuming isomalt or consuming placebo.

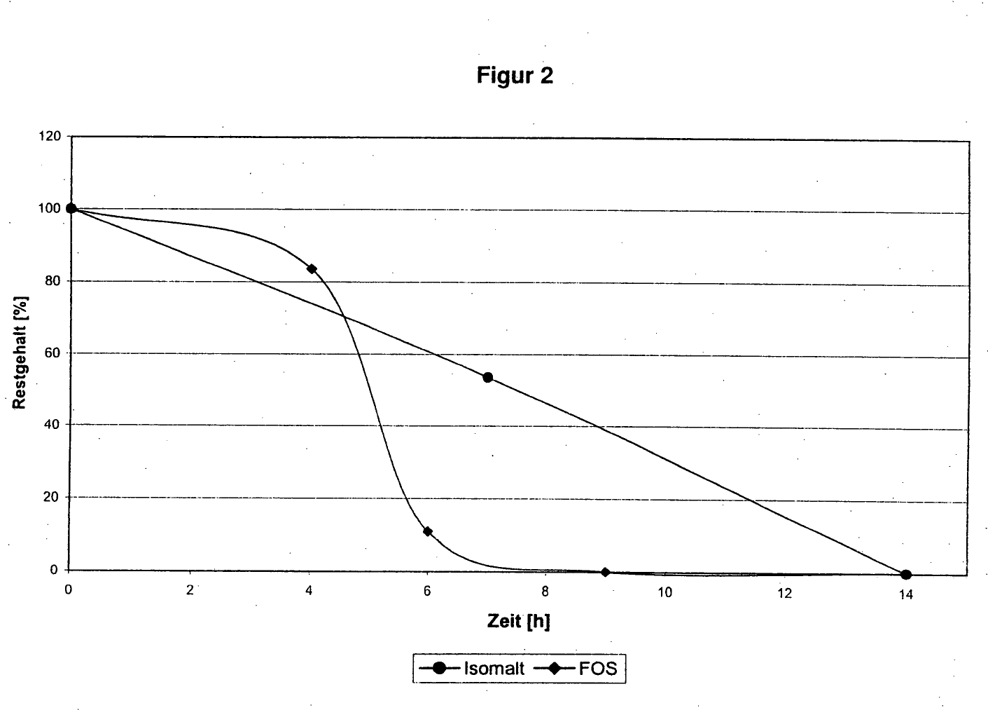

FIG. 2 shows rates of degradation of fructooligosaccharides (FOS) and isomalt on in vitro fermentation with human intestinal bacteria.

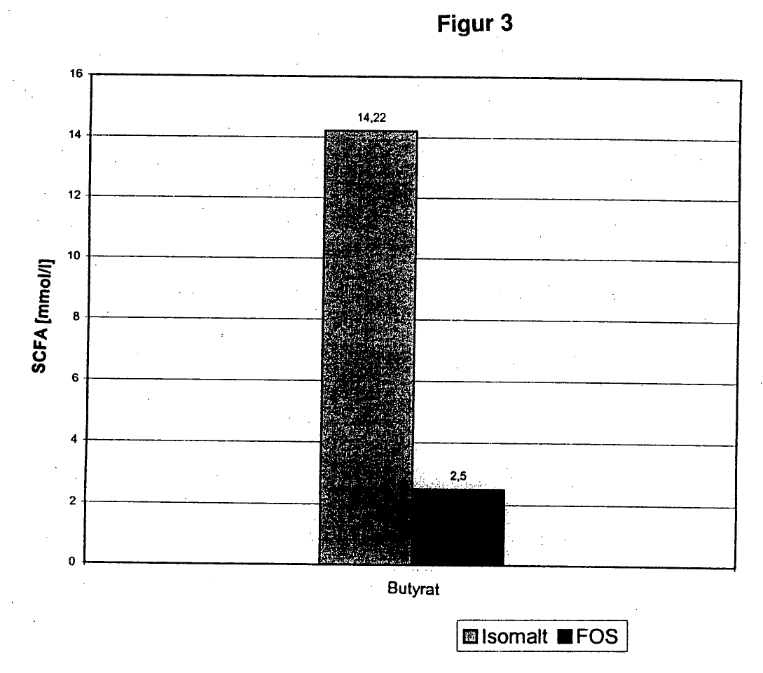

FIG. 3 depicts in the form of a histogram the formation of butyrate on in vitro fermentation of isomalt and FOS.

### EXAMPLE 1

### Effect of Isomalt on Humans

To detect the fermentative effects of isomalt on the intestinal milieu in humans and the influence on the human intestinal microflora by consuming isomalt, a human intervention study was carried out on a group of 20 healthy subjects in a double-blind, placebo-controlled crossover design. For this purpose, each of the subjects received in each case either 30 g/day isomalt as active agent or sucrose as placebo in the two 4-week test periods. The subjects received a standardized basic diet during the two test periods. The test substances were taken several times a day in the form of bakery product, jam, chocolate and other foods. The subjects received the amount of 30 g of isomalt or 30 g of placebo consumed in food products in two daily alternating planned menus. It was possible to exclude the influence of other dietary factors through an identical basic diet in both periods.

Various confectionary and bakery products were produced with isomalt and sugar:

| | | | | Amount of | |
|---|---|---|---|---|---|
| | | Amount of | | isomalt per | |
| | | sugar per day | | day | |
| Menu 1 | 30 g of jam | 7.8 | g | 7.8 | g |
| | 30 g of jam in | 7.8 | g | 7.8 | g |
| | yogurt or quark | | | | |
| | 41 g of soft | 11 | g | 11 | g |
| | biscuits | | | | |
| | 3 hard caramels | 5.4 | g | 5.4 | g |
| | (about 5.6 g) | | | | |
| | Total | 32 | g | 32 | g |
| Menu 2 | 30 g of jam | 7.8 | g | 7.8 | g |
| | 100 g of | 6.9 | g | 10 | g |
| | blancmange | | | | |
| | 1 chocolate bar + | 6.7 g + 1.8 g = 8.5 g | | 6.2 g + 1.8 g = 8 g | |
| | 1 hard caramel | | | | |
| | (1.85 g) | | | | |
| | 3 hard caramels | 5.4 | g | 5.4 | g |
| | (about 5.6 g) | | | | |
| | Total | 28.6 | g | 31.2 | g |

At the end of both test periods, the stool was collected quantitatively and, on the basis of the stool samples obtained, the qualitative and quantitative composition of the stool flora and thus also the change in individual bacteria species in relation to the total flora was determined microbiologically. The microbial stool flora without isomalt consumption was compared with the stool flora with isomalt consumption for each subject. It was thus possible to detect differences and changes due to isomalt consumption in each individual.

Analysis of the microbial stool flora took place firstly by classical microbiological diagnosis in bacteriology through the use of selective nutrient media. Secondly, analysis independent thereof took place by fluorescence in situ hybridization (FISH), a molecular biology method with fluorescence-labeled and bacterial cluster-specific RNA probes.

Microbiological Analysis Using the Nutrient Medium Technique:

Various bacterial species were investigated, including bifidobacteria and Bacteroides and Lactobacillus, in the bacteriological investigations of the stool samples.

The results of the microbiological investigations using the nutrient medium technique are shown in Tab. 1 below.

TABLE 1
Comparison of the bacterial counts in human stool
samples after consumption of 30 g/day isomalt or placebo for
four weeks

| Number of | Isomalt | | Placebo | |
|---|---|---|---|---|
| Bifidobacteria | 30 | (0.1-250)*** | 22.5 | (0.1-65) |
| Lactobacilli | 0.0004 | (0.0001-0.02)*** | 0.0003 | (0.0001-0.04) |
| Bacteroides | 32.5 | (7-300)*** | 19 | (7-325) |

The number of bacteria using the nutrient medium technique is indicated as median (Min-Max) in cfu (colony-forming units = bacterial count) × 10⁸ per g of feces;
level of significance:
***p ≦ 0.01

The bifidogenic effect of isomalt was established from comparison of the bifidobacteria in stool samples for all subjects in the study without isomalt consumption compared with stool samples with isomalt consumption.

The results of the microbiological stool investigations show for the anaerobic indicator flora that significantly more bifidobacteria were present in stool samples with isomalt consumption.

The average number of bifidobacteria in stool samples with isomalt consumption per day was more than twice as high.

Microbiological Analysis with FISH:

Fluorescence-labeled probes specific for the 16S-RNA were employed to detect bifidobacteria and the total microbe count (Eubacterium cluster) in stool samples by the FISH method (Kleessen et al. (2001), Br. J. Nutr. 86, 291-300; Schwiertz et al. (2000), Appl. Environ. Microbiol. 66, 375-381).

The results of the FISH analyses are depicted in Table 2.

TABLE 2
Number of bifidobacteria and proportion of
bifidobacteria in the total bacteria in human stool samples
after consumption of 30 g/day isomalt or placebo for four
weeks.

| | Isomalt | | Placebo | |
|---|---|---|---|---|
| Bifidobacteria [cfu × 10¹¹ | 10.3 | (0.5-42.3)* | 6.9 | (2.8-18.9) |
| per day] | | | | |
| Proportion of | 9% | (0.2-35)** | 7% | (2-14) |
| bifidobacteria in the | | | | |
| total microbe count | | | | |

The number of bacteria is indicated as the median (Min-Max) in cfu (colony-forming units = bacterial count) × 10¹¹;
level of significance:
*p ≦ 0.50;
**p ≦ 0.02

The results of the microbiological investigation of the stool samples by the FISH technique revealed significantly more bifidobacteria in stool samples per day with isomalt consumption compared with placebo (10.3 vs 6.9×10¹¹ bifidobacteria; p≦0.05). The proportion of bifidobacteria in the total bacteria in stool samples was about 30% higher with isomalt.

It was established in the microbiological investigations that consumption of isomalt-containing products leads to a significant increase in the number of bifidobacteria in stool samples and in the proportion of bifidobacteria in the total flora. Overall, the investigations established that the growth of bifidobacteria is stimulated with isomalt consumption.

These results on the increase in bifidobacteria show an improvement in the intestinal milieu with a particularly beneficial profile of microflora through isomalt consumption and thus prove the prebiotic properties of isomalt.

### EXAMPLE 2

### Effect of Isomalt on the Activity of the Microbial Enzyme β-Glucosidase

To detect the effects of isomalt on the intestinal milieu and the influence on the intestinal microflora and activity thereof in humans, as part of the intervention study described in Example 1 the activity of microbial β-glucosidase in stool samples was determined at the end of the two 4-week test periods. Detection of β-glucosidase in stool samples took place by means of a test of the cleavage of p-nitrophenyl β-D-glucopyranoside to liberate p-nitrophenol. The reaction mixture composed of 1500 μl of buffer, 400 μl of substrate (0.01 mol/l) and the stool sample was incubated at 37° C. for 1 h and, after 60 min, 1 ml of stop reagent (glycine buffer 0.1 mol/l pH 12.0) was added and the intensity of the resulting yellow coloration was determined by photometry at a wavelength of 405 nm. The intensity is proportional to the activity of the enzyme. The activity of the enzyme is indicated as liberated product [μmol] per weight [g] per unit time [h].

As depicted in FIG. 1, isomalt consumption leads to a significant reduction in the total activity of microbial β-glucosidase in stool samples. The average daily total activity of β-glucosidase was reduced by 40.3% by isomalt. The reduction in microbial β-glucosidase shows that isomalt results in an inhibition of harmful microorganisms and/or inhibition of the activity thereof. Since the liberation of potentially carcinogenic and toxic aglycones has been suggested for microbial β-glucosidase, this is regarded as a protective effect for maintaining the health of the intestine and intestinal function.

These results show an improvement in the intestinal milieu with a particularly beneficial profile of the microflora by isomalt and prove the prebiotic properties of isomalt.

### EXAMPLE 3

### Degradation of Isomalt by Bifidobacteria

In vitro investigations were carried out with pure cultures of bifidobacteria.

To investigate the growth of human bifidobacteria, various strains of human bifidobacteria (see below) were initially cultured on the following medium:

| | | |
|---|---|---|
| Caseine peptone | 10 | g |
| Meat extract | 5 | g |
| Yeast extract | 5 | g |
| Na₂HPO₄ | 1.44 | g |
| NaH₂PO₄ | 0.24 | g |
| K₂HPO₄ | 6.0 | g |
| Tween 80 | 1.0 | g |
| Cysteine/HCl | 0.5 | g |
| Trace element solution of | 9 | ml |
| DSM medium 141 | | |
| Vitamin solution of | 0.5 | ml |
| DSM medium 141 | | |
| Resazurin | 1 | mg |
| Glucose | 10 | g |

H₂O ad 1000 ml, pH 7.0

The individual strains were incubated under anaerobic conditions under an atmosphere of 80%/20% N₂/CO₂ in Hungate tubes at 37° C. for 48 h and then transferred again to the same nutrient medium. The cultures were then transferred to Hungate tubes with identical medium which contained isomalt as sole substrate. Incubation at 37° C. for a time of 48 h was followed by a second transfer to the same medium with isomalt.

Cell-free supernatants were prepared from the cultures by centrifugation at 8000×g for 15 min. The following parameters were investigated: residual isomalt content, optical density (OD₅₇₈), lactate, acetate.

Table 3 lists the results of the isomalt degradation, growth based on the increase in optical density, and the formation of lactate and acetate.

TABLE 3
Investigation of the metabolic activity of various
human bifidobacteria with isomalt

| | | | | Residual | |
|---|---|---|---|---|---|
| | | Acetate | Lactate | isomalt | Optical |
| | DSM | [mmol/ | [mmol/ | content | density |
| Species | No. | l] | l] | [%] | E₅₇₈ nm |
| B. adolescentis | 20083 | 47.9 | 23.7 | 19.7 | 1.94 |
| | 20086 | 35.7 | 27.6 | 5.5 | 1.75 |
| | 20087 | 45.9 | 20.5 | 23.5 | 2.50 |
| B. angulatum | 20098 | 46.5 | 9.9 | 29 | 2.51 |
| | 20225 | 35.4 | 4.4 | 49.7 | 2.43 |
| B. breve | 20213 | 26.1 | 2.6 | 64.3 | 1.95 |
| B. catenulatum | 20103 | 43.1 | 23.1 | 8.3 | 3.71 |
| | 20224 | 57.9 | 31.9 | 3.1 | 4.20 |
| B. infantis | 20223 | 59.8 | 21.1 | 8.5 | 3.62 |
| B. pseudocatenulatum | 20438 | 42.4 | 17.6 | 21.8 | 1.51 |

The results show that isomalt is degraded by bifidobacteria, is utilized for growth and multiplication and can lead to stimulation of lactobacteria, especially bifidobacteria, in the intestinal tract.

### EXAMPLE 4

### Comparison of the Rate of Degradation of Isomalt and Fructooligosaccharides During In Vitro Fermentation with Human Intestinal Bacteria

A 10% feces suspension in 50 mmol/l phosphate buffer, pH 7.0, was prepared under anaerobic conditions from stool samples from subjects and was employed to inoculate the following nutrient medium:

| | | |
|---|---|---|
| Tryptone | 1.5 | g |
| Yeast extract | 1.0 | g |
| KH₂PO₄ | 0.24 | g |
| Na₂HPO₄ | 0.24 | g |
| (NH₄)₂SO₄ | 1.24 | g |
| NaCl | 0.48 | g |
| MgSO₄ x 7H₂O | 0.10 | g |
| CaCl₂ x 2H₂O | 0.06 | g |
| FeSO₄ x 7H₂O | 2 | mg |
| Resazurin | 1 | mg |
| Cysteine/HCl | 0.5 | g |
| Vitamin solution (of DSM 141) | 0.5 | ml |
| Trace element solution (of DSM 141) | 9.0 | ml |
| NaHCO₃ | 2.0 | g |

Dist. H₂O ad 1000 ml, pH 7.0

To cultivate intestinal bacteria with isomalt or fructooligosaccharides, 9 ml of the detailed anaerobic medium was mixed with 0.5% (w/v) of the carbohydrate to be tested and then inoculated with 1 ml of the 10% feces suspension. Hungate tubes were incubated at 37° C. with shaking for 28 h, and samples were taken at various times and investigated for the residual carbohydrate content.

As is evident from FIG. 2, the fructooligosaccharides employed in in vitro fermentation tests were completely metabolized within about 8 h, while only after 14 h was carbohydrate no longer detectable in fermentation experiments with isomalt.

Distinctly higher concentrations of butyrate were formed (14.2 mmol/l) during the in vitro fermentation of isomalt. Only 2.5 mmol/l butyrate was synthesized on fermentation of fructooligosaccharides (FIG. 3).

The fermentative metabolism of isomalt by human intestinal flora is slower and leads to higher butyrate production than fructooligosaccharides.

### EXAMPLE 5

### Confectionery Products

Hard Caramels

| | | |
|---|---|---|
| Isomalt | 375 | g |
| Water | 120 | g |
| Citric acid | 4 | g |
| Flavor | 0.6 | g |
| Color | 0.3 | g |

Cook isomalt and water in a candy cooker at 155-160° C. Apply full vacuum for 5 min. Cool the composition to 110-115° C. Addition of acid, flavoring, coloring solution. The melt is pressed or molded.

Soft Caramels

| | | |
|---|---|---|
| Isomalt | 121 | g |
| Maltitol syrup (75% DM) | 256 | g |
| Water | 25 | g |
| Gelatine 120 Bloom (40%) | 18 | g |
| Vegetable fat (34-36° Sp) | 29 | g |
| Emulsifier | 3.8 | g |
| Citric acid (monohydrate) | 3.5 | g |
| Color (10% solution) | 0.4 | g |
| Flavor | 1 | g |

Cook isomalt, maltitol syrup and water at 132-136° C. (depending on the desired consistency). Addition of gelatin solution. Addition of vegetable fat, emulsifier, citric acid, and color in the stated sequence and mix at high speed for 2-3 minutes until a homogeneous mixture is attained. Add flavor and mix, empty the vessel. Homogenization of the composition. Cooling of the composition to 44-46° C. Allow cooled soft caramel composition to draw for 5-10 minutes (temperature then 47-49° C.) and process further.

Jellied Fruits

| | | |
|---|---|---|
| Isomalt | 152 | g |
| Lycasin | 235 | g |
| Obipektin yellow ribbon 1500 | 6.5 | g |
| Citric acid cryst. monohydr. | 2.5 | g |
| Water | 100 | g |
| Color | 0.5 | g |
| Flavor | 1 | g |

Dry mix pectin with approx. 10% of isomalt and, while stirring, sprinkle into the cold water. Bring to the boil and cook until the solution is clear. Add the remaining isomalt and Lycasin. Reduce by boiling to about 78° Brix. Add the citric acid dissolved in a little water, add the color and flavor and pour into powdered molds.

### EXAMPLE 6

### Dog Food

Dog Biscuits

| | |
|---|---|
| 150 g | quark |
| 120 g | milk |
| 90 g | sunflower oil |
| 35 g | egg yolk |
| 200 g | ground dog flakes |
| 150 g | grated cheese |
| 45 g | isomalt |

Mix the ingredients, shape into small balls and bake at 200° C. for about 20 minutes.

Dog Cookies

| | |
|---|---|
| 150 g | whole-grain wheat flour |
| 200 g | whole-grain oat flakes |
| 5 g | granulated chicken stock |
| 100 g | whole egg |
| 200 g | milk |
| 75 g | isomalt |

Mix the ingredients, roll out the dough, cut out the cookies and bake at 220° C. for about 15 minutes.

### EXAMPLE 7

### Prebiotic and Synbiotic Animal Feed Mixtures

Prebiotic Feed Mixture for Piglet Rearing

| | |
|---|---|
| Corn | 40.00 g |
| Wheat | 19.51 g |
| Extracted soybean meal | 24.36 g |
| Protex | 5.00 g |
| Soybean oil | 1.00 g |
| L-Lysine | 0.34 g |
| DL-Methionine | 0.05 g |
| Vit.-mineral feed | 2.24 g |
| Isomalt | 7.50 g |

Synbiotic Feed Mixture for Piglet Rearing

| | |
|---|---|
| Corn | 40.00 g |
| Wheat | 19.51 g |
| Extracted soybean meal | 24.36 g |
| Protex | 5.00 g |
| Soybean oil | 1.00 g |
| L-Lysine | 0.34 g |
| DL-Methionine | 0.05 g |
| Vit.-mineral feed | 2.24 g |
| Probiotic strain, e.g. | 0.01 g |
| Pediococcus acidilactici | |
| Isomalt | 7.50 g |

### EXAMPLE 8

### Muesli

Museli Bar

| | |
|---|---|
| 200 g | oat flakes |
| 100 g | corn flakes |
| 100 g | hazelnuts |
| 50 g | sunflower seeds |
| 30 g | desiccated coconut |
| 150 g | isomalt |
| 150 g | honey |
| 50 g | butter |
| 20 g | lemon juice |
| 20 g | water |

Caramelize isomalt, honey, butter, lemon juice and water. Mix oat flakes, corn flakes, nuts, sunflower seeds and desiccated coconut and add. Thoroughly mix the composition and spread on a baking sheet. Cut out bars, pack and store in a dry place.

Wintertime Fruit Muesli

| | |
|---|---|
| 80 g | oat flakes |
| 40 g | millet flakes |
| 20 g | wheatgerm flakes |
| 40 g | lemon juice |
| 150 g | yogurt |
| 20 g | sea buckthorn |
| 50 g | chopped nuts |
| 10 g | raisins |
| 400 g | apples |
| 200 g | pears |
| 300 g | oranges |
| 150 g | bananas |
| 60 g | isomalt |

Mix flakes, yogurt, sea buckthorn and nuts. Coarsely grate the apple, mix with lemon juice and add. Dice the other fruits, mix with isomalt and add.

### EXAMPLE 9

### Beverages

Power Drink

| | |
|---|---|
| 300 g | orange juice |
| 30 g | wheatgerm |
| 15 g | isomalt |
| 200 g | yogurt |

Whisk the orange juice with wheatgerm and isomalt and in the yogurt.

Sports Cocktail

| | |
|---|---|
| 250 g | carrots |
| 200 g | cucumber |
| 200 g | tomatoes |
| 250 g | apples |
| 100 g | cream |
| 10 g | parsley |
| 50 g | isomalt |

Extract juice from carrots, cucumber, tomatoes and apples. Add cream, parsley and isomalt.

Tomato Cocktail

| | |
|---|---|
| 800 g | tomatoes |
| 100 g | cream |
| 100 g | orange juice |
| 0.5 g | salt |
| 10 g | isomalt |
| 0.5 g | paprika |
| 0.5 g | Tabasco |

Purée tomatoes and mix with remaining ingredients.

### EXAMPLE 10

### Fruit Preparations

Fruit Purée

| | |
|---|---|
| Berry fruit | 230 g |
| Isomalt | 220 g |
| Binder mix | 53 g |

Flavoring and, where appropriate, coloring

Purée the fruits and bring to the boil, it being necessary to stir throughout the preparation process. Add isomalt and cook. Mix in binder mix without forming lumps. Reduce by boiling to max. 75-80% dry matter.

### EXAMPLE 11

### Dessert (Milk Product)

Dessert Cream

| | | |
|---|---|---|
| Isomalt | 334 | g |
| Skim milk powder | 110 | g |
| Corn starch | 37 | g |
| Carageenan | 13 | g |
| Vanilla flavor | 5 | g |
| Yellow color | 0.05 | g |

Thoroughly mix all the components. Stir the powder until smooth in one portion of 2500 ml of whole milk. Bring the remainder of the milk to the boil. Stir the powder mixture into the boiling milk and bring to the boil. Put into a container and store in the cool until consumed.

### EXAMPLE 12

### Jam

Südzucker Preserving Sugar Recipe

| Recipe | PS2 plus 1 | g |
|---|---|---|
| Amidated pectin | 6.4 | g |
| Citric acid | 3.8 | g |
| Sorbic acid | 0.6 | g |
| Isomalt | 489.2 | g |
| Amount of fruit | 970.0 | g |

Boiling time 4 minutes in each case

Sour Cherry Jam

| | | |
|---|---|---|
| Isomalt | 125 | g |
| Sour cherries | 225 | g |
| Pectin | 4.5 | g |
| Citric acid | 4.5 | g |
| Calcium citrate | 0.5 | g |
| L-Ascorbic acid | 0.25 | g |
| Sorbic acid | 0.25 | g |
| Water | 150 | g |

Mix pectin with ⅓ of the isomalt. Heat water with chopped cherries and pectin/isomalt mixture. Shortly before boiling add remaining amount of isomalt and the other ingredients. Boil for two minutes. Put into glass jars and fit lids.

### EXAMPLE 13

### Bakery Products

Croissants

| | |
|---|---|
| Yeast | 25.00 g |
| Cream | 300.00 g |
| Sugar | 25.00 g |
| Isomalt | 50.00 g |
| Wheat flour of type 550 | 400.00 g |
| Salt | 0.15 g |
| Margarine | 200.00 g |
| Egg yolk | 50.00 g |

Stir yeast, luke-warm cream, 1 pinch of salt and 1 pinch of flour. Leave to prove for 10 min. Knead with further ingredients and leave to prove for 20 min. Knead the dough thoroughly, roll out, cut out 15 triangles and roll up to form croissants. Leave to rise briefly and bake at 200° C. for 10 min.

White Bread

| | |
|---|---|
| Yeast | 40.0 g |
| Sugar | 15.0 g |
| Isomalt | 30.0 g |
| Wheat flour of type 550 | 1000.0 g |
| Milk | 500.0 g |
| Margarine | 250.0 g |
| Grated lemon rind | 2.5 g |
| Whole egg | 50.0 g |

Stir yeast with sugar in luke-warm milk and leave to prove for 10 min. Knead with the other ingredients and leave to prove for 20 min. Bake in a loaf tin at 175° C. for 45 min.

Sesame Bread

| | |
|---|---|
| Yeast | 60.00 g |
| Milk | 500.00 g |
| Sugar | 30.00 g |
| Isomalt | 60.00 g |
| Wheat flour of type 550 | 300.00 g |
| Rye flour of type 1150 | 250.00 g |
| Wheatmeal of type 1700 | 200.00 g |
| Salt | 0.15 g |
| Margarine | 100.00 g |
| Sesame seeds | 100.00 g |

See white bread for preparation.

Hard Cookies

| | | |
|---|---|---|
| Wheat flour of type 550 | 312 | g |
| Isomalt | 78 | g |
| Hardened peanut oil | 31 | g |
| (melting point about 35° C.) | | |
| Salt | 1.5 | g |
| Citric acid | 1.5 | g |
| (10% aqueous solution) | | |
| Milk | 70 | g |
| Ammonium bicarbonate | 3 | g |
| Sodium bicarbonate | 1.5 | g |

Suspensions of milk, isomalt, salt, citric acid and raising agent are kneaded with half of the flour to give an intermediate dough. Then preparation of the main dough from the intermediate dough, fat and remaining flour. Kneading time, intermediate dough 7 min, main dough: 13 min, dough rising time: about 20 min. Baking temperature: temperature curve of 200° C., 300° C., 270° C. Baking time about 6 min on use of a tunnel oven.

Fine Dough Without Yeast

| | | Whole-grain | |
|---|---|---|---|
| Recipe: | Soft biscuits | biscuits | |
| Wheat flour of type 550 | 51.5 g | 25.2 | g |
| Whole-grain wheatmeal | — | 25.2 | g |
| Isomalt | 15.5 g | 20 | g |
| Baking margarine, solid | 25.8 g | 20.1 | g |
| Salt | 0.3 g | 0.3 | g |
| Water | 6.7 g | 9 | g |
| Ammonium bicarbonate | 0.2 g | 0.2 | g |

Stir fats with a third of the amount of flour to give a foam, then add isomalt, salt and gradually the liquid and continue stirring until the composition is smooth. Finally, work in the remaining flour. Baking temperature: for example 200° C., about 9-13 min on use of a feed-in oven.

### EXAMPLE 14

### Milk Products

Yogurt Lemon Shake

| | |
|---|---|
| 600 g | skim-milk yogurt |
| 160 g | lemon juice |
| 60 g | honey |
| 30 g | isomalt |
| 120 g | egg yolk |

Mix ingredients

Yogurt Cream With Raspberries

| | | |
|---|---|---|
| 450 | g | whole-milk yogurt |
| 8 | g | gelatin |
| 150 | g | isomalt |
| 20 | g | lemon juice |
| 20 | g | whole milk |
| 150 | ml | cream |
| 300 | g | raspberries |

Soak the gelatin. Mix yogurt, isomalt, lemon juice and whole milk until smooth. Dissolve the gelatin and add. Beat the cream until stiff and fold into the composition. Put the raspberries into a bowl and pour yogurt composition over.

### LIST OF REFERENCES

Cummings J H, Macfarlane G T, Englyst H N. Prebiotic digestion and fermentation. Am. J. Clin. Nutr. (2001) Feb; 73 (2 Suppl):415S-420S
Kummel, K F & Brokx, S. Lactitol as functional prebiotic. Cereal Foods World (2001), 46, 425-429
Kashimura, J. Fujisawa, T. Nakajima, Y, Nishio, K, Mitsuoka, T. Utilization of palatinose, palatinose condensates, trehalulose and isomalt by various intestinal bacteria. J. Jpn. Soc. Nutr. Food. Sci. (1991), 44, 54-59.
Fuller R. Probiotics in man and animals. J Appl. Bacteriol. 1989, 66:365-378
Kleeessen B, Hartmann L, Blaut M. Oligofructose and long-chain inulin:influence on the gut microbial ecology of rats associated with a human fecel flora. Br. J. Nutr. (2001), 86(2):291-300.
Schwiertz A, Le Blay G, Blaut M. Quantification of different Eubacterium spp. in human fecal samples with species-specific 16S rRNA-targeted oligonucleotide probes. Appl. Environ. Microbiol. (2000), 66(l):375-82.

1-46. (canceled)

47. A method for forming a consumable product which comprises producing a consumable product and incorporating within said consumable product during or after said production, a mixture of 6-O-α-D-glucopyranosyl-D-sorbitol (1,6-GPS) and 1-O-α-D-glucopyranosyl-D-mannitol (1,1-GPM) in an amount and at a ratio of 1,6-GPS to 1,1-GPM sufficient to serve at least one of a prebiotic function and as a fermentable substrate in a subject upon consumption by said subject of the consumable product.

48. The method according to claim 47, wherein said mixture serves a prebiotic function such that the mixture acts as a bifidogenic prebiotic.

49. The method according to claim 47, wherein said mixture acts as a fermentable substrate such that the mixture serves as a butyrate-supplying (butyrogenic) substrate.

50. The method according to claim 47, wherein said consumable product is selected from the group consisting of a human food or other consumable product, an animal feed product and a medicament.

51. The method according to claim 50, wherein the mixture serves a prebiotic function and wherein said mixture is the sole prebiotic material contained in the consumable product.

52. The method according to claim 50, wherein the mixture serves a butyrogenic function and wherein said mixture is the sole butyrogenic material contained in the consumable product.

53. The method according to claim 47, wherein said mixture is isomalt.

54. The method according to claim 47, which further comprises adding to said mixture at least one material selected from the group consisting of mannitol, sorbitol, hydrogenated oligosaccharides, non-hydrogenated oligosaccharides and 1-O-α-D-glucopyranosyl-D-sorbitol (1,1-GPS).

55. The method according to claim 47, which further comprises adding to said mixture at least one material selected from the group consisting of a soluble dietary fiber, an insoluble dietary fiber, a fermentable dietary fiber, a non-fermentable dietary fiber and a non-digestible carbohydrate.

56. The method according to claim 55, wherein said mixture further comprises a dietary fiber and wherein the dietary fiber is formed of a material selected from the group consisting of an oligosaccharide, a polysaccharide, poly dextrose, xylorabans, inulin, a galacto-oligosaccharide, lactulose, lactitol, a xylo-oligosaccharide, lacto-sucrose, a malto-oligosaccharide, an isomalto-oligosaccharide, a gentio-oligosaccharide, glucosyl-sucrose, a soybean oligosaccharide, a chito-oligosaccharide, a chitosan-oligosaccharide, a pectin, a condensed oligosaccharide, a caramel product, a galactomannan-oligosaccharide, a fucose-containing oligosaccharide, a fucose derivative-containing oligosaccharide, modified starch, partially hydrolyzed guar gum, maltitol, sorbitol, mannitol, xylitol, erythritol, hydrogenated starch hydrolysate, pyrodextrin and variants of one or more of the above materials, obtained by a method selected from the group consisting of partial hydrolysis, hydrogenation, oxidation and enzymatic, chemical or other modification of any of them.

57. The method according to claim 56, wherein said material is an insoluble dietary fiber and wherein said insoluble dietary fiber is comprised of at least one of a resistant starch and a fiber material from at least one of wheat, oats, tomato, bean, the fruit of the locust tree, sugar beet and cellulose.

58. The method according to claim 47, wherein the mixture additionally comprises a probiotic microorganism.

59. The method according to claim 58, wherein said probiotic microorganism is derived from at least one genus selected from the group consisting of Lactobacillus and Bifidobacterium.

60. The method according to claim 47, wherein the mixture is in the form of a synbiotic.

61. The method according to claim 50, wherein said mixture is present in said consumable product as a solid or is suspended or dissolved in water in said consumable product.

62. A consumable product formed by the method according to claim 47.

63. A method for producing a human food which comprises producing a human food and incorporating within said human food, during or after said production, a mixture of 6-O-α-D-glucopyranosyl-D-sorbitol (1,6-GPS) and 1-O-α-D-glucopyranosyl-D-mannitol (1,1-GPM) in an amount and at a ratio of 1,6-GPS to 1,1-GPM sufficient to serve at least one of a prebiotic function and as a fermentable substrate in a subject upon consumption by said subject of the human food.

64. A method for producing an animal feed product which comprises producing an animal feed product and incorporating within said animal feed product, during or after said production, of a mixture of 6-O-α-D-glucopyranosyl-D-sorbitol (1,6-GPS) and 1-O-α-D-glucopyranosyl-D-mannitol (1,1-GPM) in an amount and at a ratio of 1,6-GPS to 1,1-GPM sufficient to serve at least one of a prebiotic function and as a fermentable substrate in a subject upon consumption by said subject of said animal feed product.

65. A method for producing a medicament which comprises producing a medicament and incorporating within said medicament, during or after said production, a mixture of 6-O-α-D-glucopyranosyl-D-sorbitol (1,6-GPS) and 1-O-α-D-glucopyranosyl-D-mannitol (1,1-GPM) in an amount and at a ratio of 1,6-GPS to 1,1-GPM sufficient to serve at least one of a prebiotic function and as a fermentable substrate in a subject upon consumption by said subject of the medicament.

66. A method for treating a bowel disorder in a subject in need of such treatment, which method comprises administering to said subject a therapeutically effective amount of a medicament prepared by the method of claim 65.

67. A method for achieving at least one of restoring and stabilizing a healthy intestinal flora in a subject in need of such restoration and stabilization, which method comprises administering to said subject a therapeutically effective amount of a medicament prepared by the method of claim 65.

68. A method for maintaining a relatively healthy intestinal epithelium in a subject in need of such maintenance, which method comprises administering to said subject a therapeutically effective amount of a medicament prepared by the method of claim 65.

69. A method for supporting intestinal health in a subject in need of such support, which method comprises administering to said subject a therapeutically effective amount of a medicament prepared by the method of claim 65.

70. A method for achieving at least one of restoring and promoting healthy metabolism in intestinal flora of a subject in need of said restoration or promotion, which method comprises administering to said subject a therapeutically effective amount of a medicament prepared by the method of claim 65.

71. A method for reducing toxic and harmful intestinal contents in a subject in need of such reduction, which method comprises administering to said subject a therapeutically effective amount of a medicament prepared by the method of claim 65.

72. A method for reducing oxidative stress in a subject in need of such reduction, which method comprises administering to said subject a therapeutically effective amount of a medicament prepared by the method of claim 65.

73. A method for treating a chronic inflammatory bowel disorder in a subject in need of such treatment, which method comprises administering to said subject a therapeutically effective amount of a medicament prepared by the method of claim 65.

74. A method for treating intestinal cancer in a subject in need of such treatment, which method comprises administering to such subject a therapeutically effective amount of a medicament prepared by the method of claim 65.

75. A method for treating infectious diseases in a subject in need of such treatment, which method comprises administering to such subject a therapeutically effective amount of a medicament prepared by the method of claim 65.

76. The method of claim 75, wherein the infectious disease is selected from the group consisting of bacterial intestinal infections and diarrheas.

77. A method for modulating and strengthening the immune system of a subject in need of such modulation and strengthening, which method comprises administering to such subject a therapeutically effective amount of a medicament prepared by the method of claim 65.
